(19) United States
(12) Patent Application Publication
(10) Pub. No.: US 2025/0341508 A1
(43) Pub. Date: Nov. 6, 2025

(54) SYSTEMS AND METHODS FOR DETERMINING HYDROCARBON PROPERTIES AND FRACTURE DRAINAGE HEIGHT FROM SOLID CORE SAMPLES

(71) Applicant: SAUDI ARABIAN OIL COMPANY, Dhahran (SA)

(72) Inventors: Anuj GUPTA, Katy, TX (US); Shannon EICHMANN, Katy, TX (US); Stacey ALTHAUS, Houston, TX (US); Rabah MESDOUR, Dhahran (SA); Ravimadhav N. VAIDYA, Houston, TX (US)

(73) Assignees: ARAMCO SERVICES COMPANY, Houston, TX (US); SAUDI ARABIAN OIL COMPANY, Dhahran (SA)

(21) Appl. No.: 18/655,972

(22) Filed: May 6, 2024

(51) Int. Cl. G01N 33/24

(52) U.S. Cl. CPC G01N 33/241

(57) ABSTRACT

Methods of determining an oil formation volume factor based on solid core sample data; and determining a gas/oil ratio based on the oil formation volume factor. Also, non-transitory computer-readable medium storing computer-executable instructions, which, when executed by a processor of an electronic device, cause the electronic device to: determine an oil formation volume factor based on solid core sample data; and determine a gas/oil ratio based on the oil formation volume factor

### FIELD OF THE DISCLOSURE

The present disclosure relates generally to analysis of hydrocarbon reservoirs and, more particularly, to determination of hydrocarbon type and properties in hydrocarbon reservoirs. This disclosure also relates to estimating the contributing hydraulic fracture height in wells completed with hydraulic fractures.

### BACKGROUND OF THE DISCLOSURE

Many hydrocarbon reservoirs (e.g., unconventional shale reservoirs) have significant vertical isolation between layers of geological formations within the hydrocarbon reservoir. The vertical isolation prevents fluids from moving between the layers. As a result, varying fluid types may form and be stored in different layers of the hydrocarbon reservoir, increasing complexity and cost for exploration and extraction of hydrocarbon resources.

When exploring for and extracting from hydrocarbon reservoirs, hydrocarbon properties considered when determining the potential productivity (and thus the economic value) of a hydrocarbon reservoir or region within the hydrocarbon reservoir may include the fluid type, the expected gas/oil ratio (GOR), and the condensate/gas ratio (CGR). Such factors may also need to be known for effective field development planning and for design of surface facilities.

Conventionally, the fluid type, GOR, and CGR are determined by collecting a sub-surface sample of the reservoir fluid. Alternatively, a representative sample may be collected at the surface by recombining produced hydrocarbons in an observed producing ratio. Such representative reservoir fluid samples or sub-surface samples may be used to measure the hydrocarbon properties, including oil formation volume factor, GOR, CGR, bubble-point pressure, dew-point pressure and the like, as well as to classify the reservoir fluids (e.g., black oil, volatile oil, retrograde condensate, wet gas, dry gas).

Many shale oil/gas reservoirs are developed with horizontal wells completed with one or more hydraulic fractures. Determination of the zones that are connected to and contributing to production from the horizontal well via hydraulic fractures has large implications for field development but is quite challenging.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

Some embodiments describe methods, comprising: determining an oil formation volume factor based on solid core sample data; and determining a gas/oil ratio based on the oil formation volume factor.

Other embodiments describe non-transitory computer-readable medium storing computer-executable instructions, which, when executed by a processor of an electronic device, cause the electronic device to: determine an oil formation volume factor based on solid core sample data; and determine a gas/oil ratio based on the oil formation volume factor.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a block diagram of a system for determining hydrocarbon properties in accordance with the present disclosure.

FIG. 2 is a flow chart of a method for determining hydrocarbon properties in accordance with the present disclosure.

FIG. 3 illustrates one example of a computer system that can be employed to determine hydrocarbon properties in accordance with the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to analysis of hydrocarbon reservoirs and, more particularly, to determination of hydrocarbon properties of the hydrocarbon reservoirs. The hydrocarbon properties include one or more of expected gas/oil ratio (GOR), oil formation volume factor (Bₒᵢ), condensate/gas ratio (CGR), or fluid type.

Use of the various embodiments described herein to determine hydrocarbon properties are more cost efficient than conventional methods and systems. Conventional methods and systems of evaluating hydrocarbon reservoirs and determining above hydrocarbon properties are costly due to need to obtain multiple fluid samples, as an individual sample may not accurately reflect hydrocarbon properties of the hydrocarbon reservoir. Additionally, due to the varying geologic features of the hydrocarbon reservoir, multiple samples may be required by conventional methods and systems of evaluating the hydrogen reservoir in order to obtain useful and/or accurate analysis of the hydrocarbon reservoir.

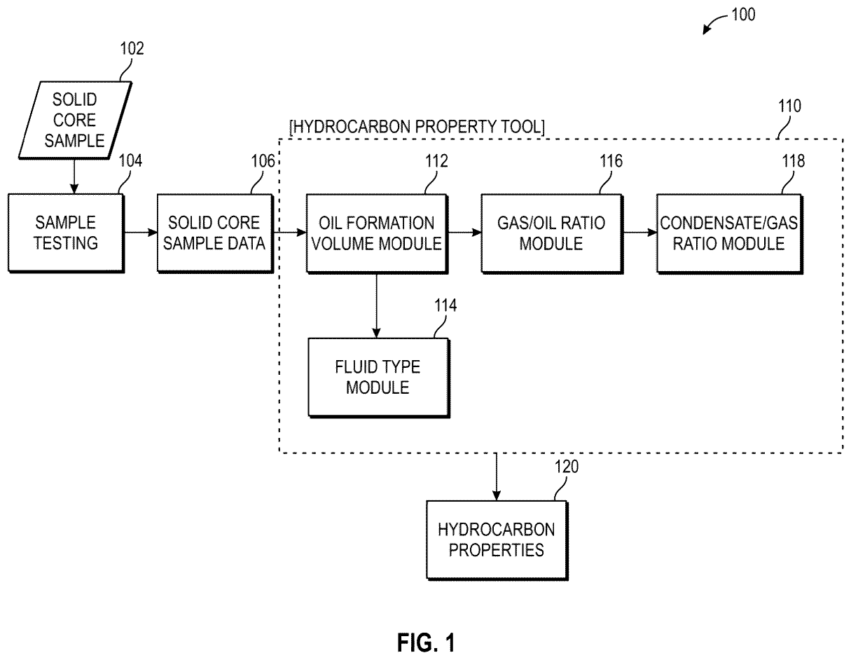

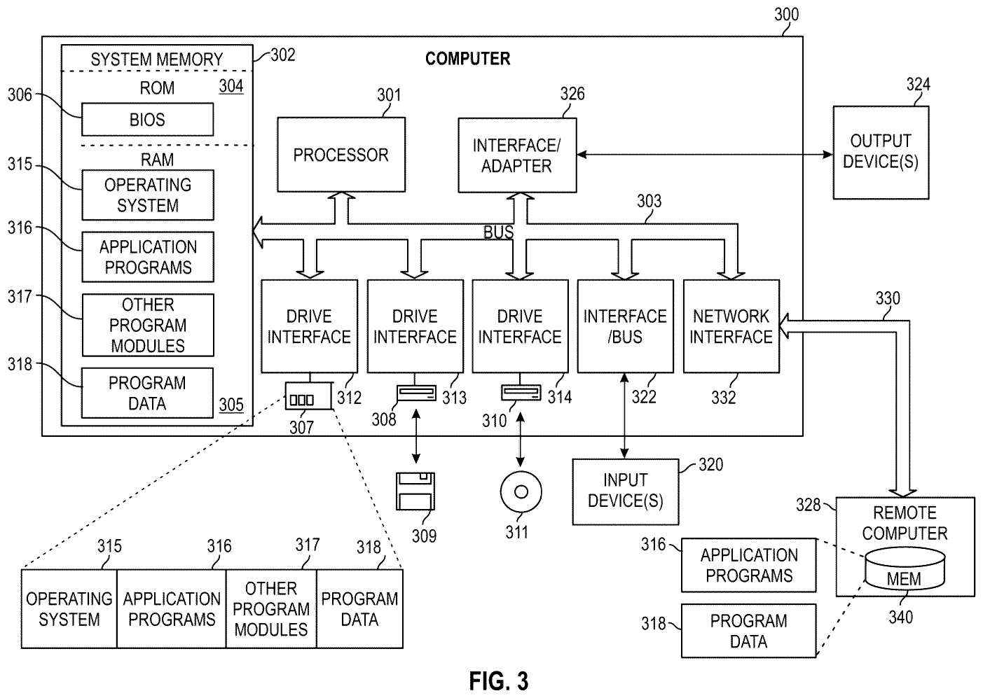

FIG. 1 is a block diagram of a system 100 for determining hydrocarbon properties 120 for hydrocarbon reservoirs. The system 100 may be partially or fully implemented by the computer system 300 of FIG. 3, for example. The system 100 enables hydrocarbon properties 120 of a hydrocarbon reservoir to be determined from solid core sample data 106.

In a non-limiting example, a solid core sample 102 of a hydrocarbon reservoir is collected. The solid core sample 102 is tested in sample testing 104 to generate solid core sample data 106. The solid core sample data 106 is an input to a hydrocarbon property tool 110 that determines hydrocarbon properties 120 of the hydrocarbon reservoir. The hydrocarbon property tool 110 includes one or more components for determining the hydrogen properties 120. In a non-limiting example, an output of an oil formation volume factor module 112 of the hydrocarbon property tool 110 is an input into one or more of a fluid type module 114 of the hydrocarbon property tool 110 or a gas/oil ratio module 116 of the hydrocarbon property tool 110. An output of the oil formation volume factor module 112 includes an oil formation volume factor for the hydrogen reservoir, for example. An output of the fluid type module 114 includes a fluid type for the hydrogen reservoir, for example. In a non-limiting example, an output of the gas/oil ratio module 116 of the hydrocarbon property tool 110 is an input into a condensate/gas ratio module 118 of the hydrocarbon property tool 110. An output of the gas/oil ratio module 116 includes a gas/oil ratio for the hydrogen reservoir, for example. An output of the condensate/gas ratio module 118 includes a condensate/gas ratio for the hydrogen reservoir, for example. The hydrocarbon property tool 110 may output one or more of the outputs of the oil formation volume factor module 112, the fluid type module 114, the gas/oil ratio module 116, or the condensate/gas ratio module 118 as the hydrocarbon properties 120.

The solid core sample 102 may comprise any solid, or at least partially solid, sample from the hydrocarbon reservoir. Examples of solid core samples may include, but are not to be limited to, a sample comprising rock, tar, mud, sand, the like, or any combination thereof. Any suitable means of obtaining the solid core sample including, but not limited to, for example, drilling, excavation, the like, or any combination thereof, may be employed. One skilled in the art will be familiar with procedures for obtaining solid core samples from a hydrocarbon reservoir. The hydrocarbon reservoir may be any suitable type of hydrocarbon reservoir including, but not limited to, a tight reservoir, or, an unconventional reservoir. “Unconventional reservoir,” and grammatical variants thereof, as used herein refers to reservoirs having hydrocarbons therein such that the hydrocarbons are tightly bound to rock within the hydrocarbon reservoir, thus requiring additional measures for exploration and excavation of said hydrocarbons.

The sample testing 104 may comprise any solid core sample test capable of generating solid core sample data 106. Example core sample data 106 may include, but are not to be limited to, GRI tests (e.g., crushed shale testing developed by Gas Research Institute), mud-rock property (MRP) tests, or like tests that generate solid core sample data 106 that includes the saturations of gas, oil, water, or any combination thereof, present in the solid core samples 102. The solid core sample data 106 includes, but is not limited to, a gas saturation, an oil saturation value, a water saturation value, or a combination thereof, for example. The gas saturation, the oil saturation, the water saturation, or a combination thereof, may indicate a degree of fluid saturation of the solid core sample 102. For example, in a solid core sample 102 having no gas saturation, an initial oil saturation may be determined using Equation 1.

$${S}_{o i} = 1 - {S}_{wi} \qquad Equation 1$$

where Sₒᵢ is the initial oil saturation and S_{wi} is the initial water saturation.

In a non-limiting example, the oil formation volume factor module 112 may use a relationship between the water saturation and oil saturation of the solid core sample data 106 to determine the oil formation volume factor (Bₒᵢ) using Equation 2. For example, the oil formation volume factor module 112 may use Equation 2 when the solid core sample data 106 does not include gas saturations

$${B}_{oi} = \frac{1 - {S}_{{w}_{solid core}}}{{S}_{{o}_{solid core}}} \qquad Equation 2$$

where Bₒᵢ is the oil formation volume factor expressed as a ratio of reservoir barrel divided by standard barrel (RB/STB), S_{w_solid core} and S_{o_solid core} are the water saturation and oil saturation, respectively, of the solid core sample data 106. When the solid core sample data 106 does not include gas saturations, the Bₒᵢ approximates the initial oil saturation because retrieval of the solid core sample 102 causes the reservoir pressure to decrease below a bubble-point pressure. In response to the decrease in reservoir pressure below the bubble-point pressure, solution-gas escapes the pore-space and the residual oil saturation reduces to a value comparable to stock-tank oil.

In a non-limiting example, the solid core sample 102 may include fluids. For example, the solid core sample 102 may include gas condensate. The sample testing 104 may include constant volume depletion (CVD) tests to determine a residual condensate saturation for a portion of the solid core sample 102 including the fluids. For example, when the oil saturations of the solid core sample data 106 is compared to a maximum liquid-drop-off determined by a CVD test, the oil formation volume factor module 112 yields apparent Bₒᵢ. Apparent Bₒᵢ may be larger than a Bₒᵢ valid for an oil reservoir, indicating that the example reservoir is a gas-condensate reservoir.

In a non-limiting example, the fluid type module 114 uses the oil formation volume factor to determine a fluid type within the hydrocarbon reservoir. Determining fluid type may include categorization of the fluid due to the value of the oil formation volume factor. The fluid type module 114 may use data of a standards or specification associated with hydrocarbon reservoirs. The data may be from a classification of petroleum fluids provided by a professional organization of petroleum engineers, for example. As a non-limiting example, the fluid type may comprise heavy oils and tars, black oils, volatile oils, gas condensates, or wet and dry gases. For example, if the oil formation volume factor is between 1.1 and 1.5, the fluid type module 114 may determine that the fluid type is black oil; if the oil formation volume factor is between 1.5 to 3.0, the fluid type module 114 may determine the fluid type is volatile oil; or if the oil formation volume factor is between 3.0 and 20.0, the fluid type module 114 may determine the fluid type is gas condensate.

In a non-limiting example, the gas/oil ratio module 116 uses the oil formation volume factor (Bₒᵢ) output by the oil formation volume factor module 112 to determine a gas/oil ratio. Many methods of determining gas/oil ratio from the oil formation volume factor are available. For example, the gas/oil ratio module 116 may use Equation 3 and 4 below. Reference is made to McCain, William D. The Properties of Petroleum Fluids, 2nd. Ed. (Eq. B-47 and 48, pg. 522).

$$CN = {( \frac{{B}_{oi} - 0.9759}{0.00012} )}^{\frac{1}{1.2}} \qquad Equation 3$$

where Bₒᵢ is the oil formation volume factor (RB/STB) and CN is a unitless parameter.

$$GOR = \frac{CN - 1.25 T}{\sqrt{\frac{{SG}_{g a s}}{{SG}_{o i l}}}} \qquad Equation 4$$

where GOR defines the gas/oil ratio (standard cubic feet/stock tank barrel or SCF/STB), CN is a unitless parameter, SG_{gas} and SGₒᵢₗ are the specific gravities of gas (with respect to air) and oil (with respect to water), respectively, produced from the hydrocarbon reservoir in question, and T is the temperature of the hydrocarbon reservoir in degrees Fahrenheit (° F.). The specific gravities of gas and oil may each be obtained from the samples of produced fluids from the candidate well, offset wells of the hydrocarbon reservoir or any suitable source.

In a non-limiting example, the condensate/gas ratio module 118 uses the GOR (SCF/STB) output by the gas/oil ratio module 116 to determine a condensate/gas ratio (stock tank barrel per million standard cubic feet or STB/MMSCF). Many methods of determining condensate/gas ratio from the oil formation volume factor are available. For example, the amount of gas dissolved in a hydrocarbon reservoir may conventionally be represented in standard cubic feet (SCF) per stock-tank barrel (STB) and is thus expressed as the gas/oil ratio GOR with units of SCF/STB. Conversely a stock-tank barrel (STB) of condensate produced for every million standard cubic feet (MMSCF) may be expressed at the condensate/gas ratio CGR with units of STB/MMSCF. Thus, Equation 5a can be used to relate GOR and CGR.

$$GOR = \frac{1 , 000 , 000}{CGR} \qquad Equation 5 a$$

where GOR is expressed in SCF/STB and CGR is expressed in STB/MMSCF. Thus, reciprocally the relationship may also be expressed through Equation 5b.

$$CGR = \frac{1 , 000 , 000}{GOR} \qquad Equation 5 b$$

### METHOD(S)

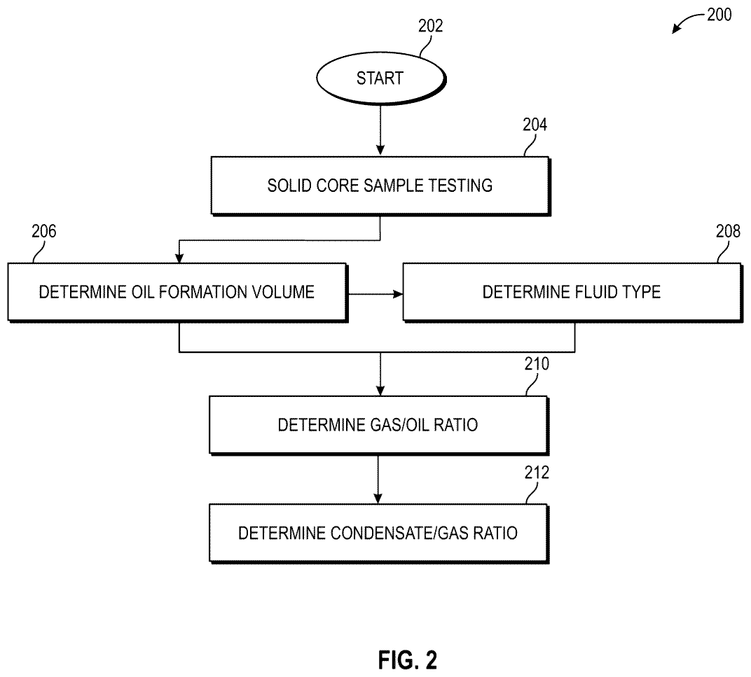

In view of the structural and functional features described above, example methods will be better appreciated with reference to FIG. 2. FIG. 2 is a flow chart of a method 200 for determining hydrocarbon properties, in accordance with certain embodiments. The method 200 may be performed by a system for determining hydrocarbon properties. The method 200 may be performed by the system 100 of FIG. 1, for example. The hydrocarbon properties may be the hydrocarbon properties 120, for example. The method 200 includes starting (block 202), solid core sample testing (block 204), determining oil formation volume (block 206), determining fluid type (block 208), determining gas/oil ratio (block 210), and determining condensate/gas ratio (block 212).

The method 200 starts at block 202. The method 200 may start in response to receiving an input from a user, a system described herein, or another system communicatively coupled to the system described herein. In a non-limiting example, the method 200 starts at the block 202 in response to an indication that a solid core sample is available. The solid core sample may be the solid core sample 102 of FIG. 1, for example. The method 200 includes testing the solid core sample at block 204. Testing the solid core sample may include generating solid core sample data. The solid core sample data may be the solid core sample data 106, for example. The method 200 further includes determining the oil formation volume at block 206 based on the solid core sample data. Determining the oil formation volume may use one or more techniques as described with respect to the oil formation volume module 112 of FIG. 1, for example. The method 200 includes determining fluid type at block 208 based on the oil formation volume factor generated at block 206. Determining the fluid type may use one or more techniques as described with respect to the fluid type module 114 of FIG. 1, for example. The method 200 furthermore includes determining a gas/oil ratio at block 210 based on the oil formation volume factor generated in block 206. Determining the gas/oil ratio may use one or more techniques as described with respect to gas/oil ratio module 116 of FIG. 1, for example. The method 200 furthermore includes determining a condensate/gas ratio at block 212 based on the gas/oil ratio generated at block 210. Determining the condensate/gas ratio may use one or more techniques as described with respect to the condensate/gas ratio module 118 of FIG. 1, for example. Within the method 200, any combination of the determined hydrocarbon properties may be outputted to a user, transferred to another system, the like, or any combination thereof.

While, for purposes of simplicity of explanation, the example methods of FIG. 2 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

### EXAMPLE(S)

As a nonlimiting example, solid core samples were obtained, and example hydrocarbon properties were generated using the system 100 of FIG. 1 and the method 200 of FIG. 2. Oil saturation and water saturation values were used to generate an apparent oil formation volume factor as described in the present disclosure. A fluid type was determined based on the apparent oil formation volume factor. GOR and CGR were generated based on the apparent oil formation volume factor. The reservoir temperature, T, was assumed to be 277° F. and the specific gravity of gas, SG_{gas}, and of oil, SGₒᵢₗ, were assumed to be 1.1 and 0.78, respectively. Results of example calculations are visible in Table 1 below.

TABLE 1
Example of hydrocarbon properties of a hydrocarbon reservoir.

| | | | Apparent Oil | | | |
|---|---|---|---|---|---|---|
| | Oil | Water | Formation | | | |
| Sample | Saturation | Saturation | Volume Factor | | GOR (SCF/ | CGR |
| ID | (Sₒ) | (S_{w}) | (Bₒᵢ) (RB/STB) | Fluid Type | STB) | (STB/MMSCF) |
| A1 | 61.94 | 29.33 | 1.14 | Black Oil | 54 | 18519 |
| A2 | 67.88 | 8.09 | 1.35 | Black Oil | 394 | 2538 |
| A3 | 52.51 | 11.94 | 1.68 | Volatile Oil | 870 | 1149 |
| A4 | 48 | 4.13 | 2.00 | Volatile Oil | 1295 | 772 |
| A5 | 40.57 | 3.74 | 2.37 | Volatile Oil | 1760 | 568 |
| A6 | 26.66 | 5.82 | 3.53 | Gas | 3105 | 322 |
| | | | | Condensate | | |
| A7 | 15.87 | 6.67 | 5.88 | Gas | 5558 | 180 |
| | | | | Condensate | | |
| A8 | 47.72 | 7.21 | 1.94 | Volatile Oil | 1217 | 822 |

As another nonlimiting example, solid core samples were obtained, and example hydrocarbon properties were generated using the system 100 of FIG. 1 and the method 200 of FIG. 2. Oil saturation and water saturation values were used to generate apparent oil formation volume factor as described in the present disclosure. GOR and CGR were generated based on the apparant oil formation volume factor. The reservoir temperature, T, was assumed to be 277° F. and the specific gravity of gas, SG_{gas}, and of oil, SGₒᵢₗ, were assumed to be 1.1 and 0.78, respectively. Results of example calculations are visible in Table 2 below.

TABLE 2
Example of hydrocarbon properties of a hydrocarbon reservoir.

| | | | Apparent Oil | | | |
|---|---|---|---|---|---|---|
| | Oil | Water | Formation | | GOR | |
| Sample | Saturation | Saturation | Volume Factor | | (SCF/ | CGR |
| ID | (Sₒ) | (S_{w}) | (Bₒᵢ) (RB/STB) | Fluid Type | STB) | (STB/MMSCF) |
| B1 | 21.54 | 42.71 | 2.66 | Volatile Oil | 2109 | 474 |
| B2 | 12.04 | 45.40 | 4.53 | Gas | 4187 | 239 |
| | | | | Condensate | | |
| B3 | 19.50 | 54.46 | 2.33 | Volatile Oil | 1717 | 583 |
| B4 | 17.49 | 66.34 | 1.92 | Volatile Oil | 1197 | 836 |
| B5 | 22.26 | 35.66 | 2.89 | Volatile Oil | 2380 | 420 |
| B6 | 15.10 | 31.84 | 4.52 | Gas | 4166 | 240 |
| | | | | Condensate | | |
| B7 | 8.79 | 71.22 | 3.28 | Gas | 2821 | 354 |
| | | | | Condensate | | |
| B8 | 35.40 | 18.26 | 2.31 | Volatile Oil | 1685 | 593 |
| B9 | 10.74 | 26.86 | 6.81 | Gas | 6469 | 155 |
| | | | | Condensate | | |
| B10 | 7.92 | 50.52 | 6.24 | Gas | 5918 | 169 |
| | | | | Condensate | | |
| B11 | 26.72 | 21.79 | 2.93 | Volatile Oil | 2423 | 413 |
| B12 | 11.71 | 34.75 | 5.57 | Gas | 5251 | 190 |
| | | | | Condensate | | |
| B13 | 18.36 | 20.95 | 4.31 | Gas | 3945 | 253 |
| | | | | Condensate | | |
| B14 | 8.79 | 24.80 | 4.44 | Gas | 4086 | 245 |
| | | | | Condensate | | |
| B15 | 17.49 | 22.36 | 4.95 | Gas | 4618 | 217 |
| | | | | Condensate | | |
| B16 | 17.81 | 11.88 | 2.03 | Volatile Oil | 1330 | 752 |
| B17 | 39.28 | 20.38 | 3.04 | Gas | 2555 | 391 |
| | | | | Condensate | | |
| B18 | 25.93 | 21.12 | 3.23 | Gas | 2774 | 360 |
| | | | | Condensate | | |

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 3. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 3 illustrates one example of a computer system 300 that can be employed to execute one or more embodiments of the present disclosure. Computer system 300 can be implemented on one or more general purpose networked computer systems, embedded computer systems, routers, switches, server devices, client devices, various intermediate devices/nodes or standalone computer systems. Additionally, computer system 300 can be implemented on various mobile clients such as, for example, a personal digital assistant (PDA), laptop computer, pager, and the like, provided it includes sufficient processing capabilities.

Computer system 300 includes processing unit 302, system memory 304, and system bus 306 that couples various system components, including the system memory 304, to processing unit 302. System memory 304 can include volatile (e.g. RAM, DRAM, SDRAM, Double Data Rate (DDR) RAM, etc.) and non-volatile (e.g. Flash, NAND, etc.) memory. Dual microprocessors and other multi-processor architectures also can be used as processing unit 302. System bus 306 may be any of several types of bus structure including a memory bus or memory controller, a peripheral bus, and a local bus using any of a variety of bus architectures. System memory 304 includes read only memory (ROM) 310 and random access memory (RAM) 312. A basic input/output system (BIOS) 314 can reside in ROM 310 containing the basic routines that help to transfer information among elements within computer system 300.

Computer system 300 can include a hard disk drive 316, magnetic disk drive 318, e.g., to read from or write to removable disk 320, and an optical disk drive 322, e.g., for reading CD-ROM disk 324 or to read from or write to other optical media. Hard disk drive 316, magnetic disk drive 318, and optical disk drive 322 are connected to system bus 306 by a hard disk drive interface 326, a magnetic disk drive interface 328, and an optical drive interface 330, respectively. The drives and associated computer-readable media provide nonvolatile storage of data, data structures, and computer-executable instructions for computer system 300. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 310, including operating system 332, one or more application programs 334, other program modules 336, and program data 338. In some examples, the application programs 334 can include hydrocarbon property tool 110 of FIG. 1, and the program data 338 can include one or more hydrocarbon properties (e.g., oil formation volume factor, fluid type, gas/oil ratio, condensate/gas ratio). The application programs 334 and program data 338 can include functions and methods programmed to determine hydrocarbon properties, such as shown and described herein.

A user may enter commands and information into computer system 300 through one or more input devices 340, such as a pointing device (e.g., a mouse, touch screen), keyboard, microphone, joystick, game pad, scanner, and the like. For instance, the user can employ input device 340 to edit or modify solid core sample data and/or other like input data and/or data. These and other input devices 340 are often connected to processing unit 302 through a corresponding port interface 342 that is coupled to the system bus, but may be connected by other interfaces, such as a parallel port, serial port, or universal serial bus (USB). One or more output devices 344 (e.g., display, a monitor, printer, projector, or other type of displaying device) is also connected to system bus 306 via interface 346, such as a video adapter.

Computer system 300 may operate in a networked environment using logical connections to one or more remote computers, such as remote computer 348. Remote computer 348 may be a workstation, computer system, router, peer device, or other common network node, and typically includes many or all the elements described relative to computer system 300. The logical connections, schematically indicated at 350, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 300 can be connected to the local network through a network interface or adapter 352. When used in a WAN networking environment, computer system 300 can include a modem, or can be connected to a communications server on the LAN. The modem, which may be internal or external, can be connected to system bus 306 via an appropriate port interface. In a networked environment, application programs 334 or program data 338 depicted relative to computer system 300, or portions thereof, may be stored in a remote memory storage device 354.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

What is claimed is:

1. A method, comprising:
determining an oil formation volume factor based on solid core sample data; and
determining a gas/oil ratio based on the oil formation volume factor.

2. The method of claim 1, further comprising determining a condensate/gas ratio based on the gas/oil ratio.

3. The method of claim 1, wherein determining the condensate/gas ratio comprises multiplying the reciprocal of the gas/oil ratio by 1,000,000.

4. The method of claim 1, wherein determining the oil formation volume factor based on the solid core sample data comprises:
receiving a water saturation value based on the solid core sample data;
receiving an oil saturation value based on the solid core sample data; and
determining the oil formation volume factor based on the water saturation value and the oil saturation value.

5. The method of claim 1, further comprising selecting a fluid type based on the oil formation volume factor.

6. The method of claim 5, wherein the fluid type comprises black oil, volatile oil, or gas condensate.

7. The method of claim 5, wherein:
a) the oil formation volume factor is less than 1.5 and the fluid type is black oil;
b) the oil formation volume factor is from 1.5 to 3.0 and the fluid type is volatile oil; or
c) the oil formation volume factor is greater than 3.0 and the fluid type is gas condensate.

8. The method of claim 1, wherein determining the gas/oil ratio based on the oil formation volume factor comprises:
determining a parameter based on the oil formation volume factor; and
determining the gas/oil ratio based on the parameter, a temperature, a gas specific gravity, and an oil specific gravity.

9. The method of claim 1, wherein the solid core sample data comprises GRI test data, MRP test data, or any combination thereof.

10. The method of claim 1, further comprising:
obtaining a solid core sample from a hydrocarbon reservoir; and
generating the solid core sample data based on the core sample.

11. The method of claim 10, wherein the hydrocarbon reservoir comprises an unconventional reservoir.

12. A non-transitory computer-readable medium storing computer-executable instructions, which, when executed by a processor of an electronic device, cause the electronic device to:
determine an oil formation volume factor based on solid core sample data; and
determine a gas/oil ratio based on the oil formation volume factor.

13. The non-transitory computer-readable medium of claim 12, further comprising determining a condensate/gas ratio based on the gas/oil ratio.

14. The non-transitory computer-readable medium of claim 12, wherein determining the condensate/gas ratio comprises multiplying the reciprocal of the gas/oil ratio by 1,000,000.

15. The non-transitory computer-readable medium of claim 12, wherein determining the oil formation volume factor based on the solid core sample data comprises:
receiving a water saturation value based on the solid core sample data;
receiving an oil saturation value based on the solid core sample data; and
determining the oil formation volume factor based on the water saturation value and the oil saturation value.

16. The non-transitory computer-readable medium of claim 12, further comprising selecting a fluid type based on the oil formation volume factor.

17. The non-transitory computer-readable medium of claim 16, wherein the fluid type comprises black oil, volatile oil, or gas condensate.

18. The non-transitory computer-readable medium of claim 16, wherein:
a) the oil formation volume factor is less than 1.5 and the fluid type is black oil;
b) the oil formation volume factor is from 1.5 to 3.0 and the fluid type is volatile oil; or
c) the oil formation volume factor is greater than 3.0 and the fluid type is gas condensate.

19. The non-transitory computer-readable medium of claim 12, wherein generating the gas/oil ratio based on the oil formation volume factor comprises:
determining a parameter based on the oil formation volume factor; and
determining the gas/oil ratio based on the parameter, a temperature, a gas specific gravity, and an oil specific gravity.

20. The non-transitory computer-readable medium of claim 12, wherein the solid core sample data comprises GRI test data, MRP test data, or any combination thereof.
